(19) United States
(12) Patent Application Publication
(10) Pub. No.: US 2006/0024341 A1
(43) Pub. Date: Feb. 2, 2006

(54) Microemulsion composition and related method

(76) Inventor: William J. Roberts, Ocala, FL (US)

Correspondence Address: Stephen T. Sullivan, Suite 1140, 1850 N. Central Avenue, Phoenix, AZ, 85004-4586, US

(21) Appl. No.: 10/910,548

(22) Filed: Aug. 2, 2004

(51) Int. Cl. A61K 8/39

(52) U.S. Cl. 424/401; 424/70.28

(57) ABSTRACT

A composition for oral administration to a subject as a microemulsion. The composition includes a surfactant having a hydrophilic-lipophilic balance of between about 12 and 16, a cosurfactant having a hydrophilic-lipophilic balance of between about 5 and 6, and an active ingredient, wherein the composition comprises a microemulsion when added to water. Related compositions and methods of administration are provided as well.

### BACKGROUND OF THE INVENTION

1. Field of the Invention

The present invention relates to biochemistry and pharmacology and, more specifically, to compositions and methods for improving solubility and/or bioavailability of active ingredients, particularly when administered orally, including sublingually.

2. Description of the Related Art

When administering a particular active ingredient or combination of active ingredients, whether it be a pharmaceutical composition, a nutraceutical composition, a dietary supplement, or combinations of these, to a subject, it is very important that the desired amount of the active ingredient or ingredients be absorbed into the system. This is true whether the subject is a human, as well as in veterinary applications. One usually desires to maximize the amount of the active ingredient or ingredients that are available for absorption into the system of the subject (the amount provided), and the amount of the active ingredient or ingredients that actually are absorbed (bioavailability). Incidentally, the term “active ingredient” as used herein below will be used to refer to the ingredient or ingredients that are desired to be administered to the subject and desired to be absorbed into the system of the subject.

Administration of an active ingredient can be greatly facilitated where it can be orally administered, for example, for direct ingestion into the alimentary tract, sublingually, etc. This normally provides better absorption and bioavailability than such administration routes as percutaneous administration, and is less burdensome that intravenous administration.

The objectives of enabling oral administration while optimizing the desired bioavailability and absorption are undermined, however, when the active ingredient has low solubility. To address this limitation, it is common with such preparations to package or contain them in capsules or gel caps. The problem persists, however, when the active ingredient is insoluble, or has low solubility in the liquid phase of the capsule or gel cap. The problem is even more limiting when a substantial amount of the active ingredient is required or desired to be absorbed by the subject. These limitations can require that the subject take multiple capsules or doses, which can be problematic from a practical perspective.

One approach to address these limitations has involved the use of excipients. Examples may be found in the literature and in a number of issued patents.

Another approach has involved the use of solubilizing agents. Examples of this approach include, for example, the compositions and methods disclosed in U.S. Pat. No. 6,054,136 and No. 6,312,704. These patents, and others like them, disclose the use of solubilizing agents that can be used to create microemulsions. The microemulsions improve the solubility of the active ingredient, for example, by containing it in the microemulsion and thereby preventing the relatively insoluble active ingredient from agglomerating into larger, insoluble separate phases. These approaches, while improving solubility concerns, also have been limited, however, for example, in that they still do not afford satisfactory bioavailability in many instances. This is particularly true with active ingredients that are poorly soluble in oils, poorly soluble or essentially insoluble in water, wherein they have low bioavailability, which have relatively low molecular weight (e.g., between 100 and 1,000), and/or which have a relatively low melting point (e.g., between about 60° C. and 250° C.

### OBJECTS OF THE INVENTION

Accordingly, an object of the present invention is to provide compositions and methods to facilitate the solubility of active ingredients.

Another object of the invention is to provide compositions and methods that facilitate the creation and stabilization of microemulsions to improve solubility and/or bioavailability.

Another object of the invention is to provide compositions and methods that facilitate solubility and bioavailability of active ingredients.

Additional objects and advantages of the invention will be set forth in the description which follows, and in part will be apparent from the description, or may be learned by practice of the invention. The objects and advantages of the invention may be realized and obtained by means of the instrumentalities and combinations pointed out in the appended claims.

### SUMMARY OF THE INVENTION

To achieve the foregoing objects, and in accordance with the purposes of the invention as embodied and broadly described in this document, a microemulsion composition comprising. It comprises a surfactant having a hydrophilic-lipophilic balance (“HLB”) of between about 12 and about 16, and a cosurfactant having a hydrophilic-lipophilic balance of between about 5 and about 6. This comprises a “microemulsion composition” in that, when added to water, it creates a microemulsion. The use of the term “microemulsion composition” as used throughout this document is used in this sense, to include compositions that, when added to water, form a microemulsion.

“Surfactant” as the term is used herein is used according to its ordinary meaning in the field, for example, to mean a surface active agent having polar or ionic portion and a non-polar, typically aliphatic portion. This dual functionality of the molecule permits the surfactant to form micelles, which allow them to be reasonably soluble in water, but yet to encapsulate and solubilize non-polar substances, such as fats or other organics.

In some presently preferred embodiments of the invention, the surfactant has an HLB value about 12 to about 14. In others, the surfactant has an HLB value of about 14 to about 16. In still others, the surfactant has an HLB value of 14 to about 15. In some of the specific embodiments that are presently preferred, the surfactant has an HLB value of about 14, and in others about 15.

Examples of surfactants that would be suitable for use in accordance with various aspects of the invention are certain polyoxyl castor oils, glycerol polyethylene glycol ricinoleate, glycerol polyethylene glycol oxystearate, polyethylene glycol-15-hydroxystearate, and the like.

“Cosurfactant” also is used herein according to its ordinary meaning in the field, to include, for example, substances that function as co-surface active agents, i.e., wherein they have hydrophobic character, and facilitate mutual solubilization of polar and non-polar phases in a microemulsion.

In presently preferred embodiments of the invention, the cosurfactant has an HLB value of about 5, and in others about 6. Examples of presently preferred cosurfactants according to presently preferred embodiments include propylene glycol monocaprylate.

The relative amounts of the surfactant and the cosurfactant may differ in various embodiments according to the invention, as presently preferred. To facilitate an understanding of these relative proportions, and to illustrate, the surfactant and the cosurfactant together have a combined mass, which in most cases will be the sum of their individual masses. In some presently preferred embodiments, the surfactant comprises about 30% to 70% of this combined mass, and/or the cosurfactant comprises about 30% to 70% of the combined mass.

In preferred embodiments according to this aspect of the invention, the microemulsion composition is free or substantially free of a lipid phase.

In accordance with another aspect of the invention, a composition is provided for oral administration to a subject. The composition comprises a surfactant having an HLB value of between about 12 and 16, a cosurfactant having an HLB value of between about 5 and 6, and an active ingredient (including combinations of active ingredients), wherein this composition comprises a microemulsion when added to water.

The surfactant and cosurfactant may be as described herein for the previously described embodiments.

The active ingredient according to this aspect of the invention may comprise any active ingredient (including combinations thereof) that are desired to be administered to the subject, and particularly those that suffer the limitations of solubility, absorptivity and/or bioavailability as described herein above. Examples of active ingredients accordingly to presently preferred embodiments of the invention would include androst-1-ene-3-one-17b-ethylcarbonate, androst-4-ene-3,17b-di(ethylcarbonate), forskolin 1,9-carbonate, sclareolide, androst-5-ene-7-one-3,17b-(di)ethylcarbonate), valerian root or extract, Tribulus terrestris extract, and the like.

In accordance with another aspect of the invention, a composition is provided for oral administration to a subject. The composition comprises a surfactant having a hydrophilic-lipophilic balance of between about 12 and 16, a cosurfactant having a hydrophilic-lipophilic balance of between about 5 and 6, and an active ingredient comprising androst-1-ene-3-one-17b-ethylcarbonate, wherein the composition comprises a microemulsion when added to water.

In accordance with still another aspect of the invention, a composition is provided for oral administration to a subject. The composition comprises a surfactant having a hydrophilic-lipophilic balance of between about 12 and 16, a cosurfactant having a hydrophilic-lipophilic balance of between about 5 and 6, and an active ingredient comprising androst-4-ene-3,17b-di(ethylcarbonate), wherein the composition comprises a microemulsion when added to water.

In accordance with yet another aspect of the invention, a composition is provided for oral administration to a subject. The composition comprises a surfactant having an HLB value of between about 12 and 16, a cosurfactant having an HLB value of between about 5 and 6, and an active ingredient comprising androst-1-ene-3-one-17b-ethylcarbonate and androst-4-ene-3, 17b-di(ethylcarbonate), wherein the composition comprises a microemulsion when added to water.

In each of these aspects of the invention, the surfactant and cosurfactant may be as described herein for the embodiments previously described above. In the presently preferred embodiments according to these aspects of the invention, the surfactant comprises a polyoxyl castor oil, and/or glycerol polyethylene glycol ricinoleate, glycerol polyethylene glycol oxystearate, polyethylene glycol-15-hydroxystearate, or combinations of these. In these presently preferred embodiments, the cosurfactant comprises propylene glycol monocaprylate. The surfactant constitutes about 40% of the combined volume and the cosurfactant comprising about 60% of the combined volume, or, equivalently, the surfactant constitutes about 47% of the combined mass and the cosurfactant comprising about 53% of the combined mass.

In accordance with a further aspect of the invention, a composition is provided for oral administration to a subject. The composition comprises a surfactant having an HLB value of between about 12 and 16, a cosurfactant having an HLB value of between about 5 and 6, and an active ingredient comprising forskolin 1,9-carbonate, wherein the composition comprises a microemulsion when added to water.

In accordance with still another aspect of the invention, a composition is provided for oral administration to a subject. This composition comprises a surfactant having an HLB value of between about 12 and 16, a cosurfactant having a hydrophilic-lipophilic balance of between about 5 and 6, and an active ingredient comprising sclareolide, wherein the composition comprises a microemulsion when added to water.

In accordance with another aspect of the invention, a composition is provided for oral administration to a subject. The composition comprises a surfactant having an HLB of between about 12 and 16, a cosurfactant having an HLB value of between about 5 and 6, and an active ingredient comprising androst-5-ene-7-one-3,17b-(di)ethylcarbonate), wherein the composition comprises a microemulsion when added to water.

In accordance with a still further aspect of the invention, a method is provided for orally administering a composition to a subject. The method comprises providing the composition, wherein the composition comprises a surfactant having an HLB value of between about 12 and 14, a cosurfactant having an HLB value of between about 5 and 6, and an active ingredient, and adding water to form a microemulsion. The method also includes administering the composition, i.e., the microemulsion, to the subject orally, preferably in a capsule containing the composition in microemulsified form.

### DETAILED DESCRIPTION OF THE PREFERRED EMBODIMENTS AND METHODS

Reference will now be made in detail to the presently preferred embodiments and methods of the invention as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the preferred embodiments and methods. The invention according to its various aspects is particularly pointed out and distinctly claimed in the attached claims read in view of this specification, and appropriate equivalents.

In accordance with one aspect of the invention, a microemulsion (or microemulsifying) composition is provided that comprises a surfactant having an HLB value of between about 12 and about 16, and a cosurfactant having an HLB value of between about 5 and about 6.

As noted above, the surfactant may comprise a number of different surface active agents, alone or in combination. The HLB value or values of the surfactant may depend upon the specific application. In some preferred embodiments, the HLB value for the surfactant or surfactant combination ranges between about 12 to about 14. In others, it ranges between about 14 to about 16. In still others, they range between about 14 to about 15. In some embodiments and applications, the HBL value of the surfactant or surfactant combination is about 14 and in some it is about 15. The HLB value of the surfactant or surfactant combination preferably is selected so that the surfactant and the system (i.e., the surfactant, cosurfactant and if present, the active ingredient) are solubilized in the capsule or other desired packaging, container or delivery system with water to form a microemulsion, and the microemulsion is sufficiently bioavailable. The specific HLB value preferably is selected so that the system has satisfactory solubility while being substantially clear, e.g., substantially translucent or transparent to the naked eye when added to water and particularly when added to water that is at a temperature of about 37 C.

Presently preferred surfactants comprise a polyoxyl castor oil, or a polyethoxylated caster oil, preferably comprising glycerol polyethylene glycol ricinoleate. An example of such surfactant would be CREMOPHOR® EL, commercially available from BASF Aktiengesellschaft, Fine Chemicals Division, Germany (“BASF”). This surfactant may comprise a hydrophobic portion such as fatty acid esters of polyethylene glycol, and a hydrophilic portion such as free polyethylene glycols and ethoxylated glycerol, both of which are reported included in CREMOPHOR® EL.

The surfactant also may comprise a polyoxyl caster oil comprising glycerol polyethylene glycol oxystearate. An example of such surfactant would be CREMOPHOR® RH 40, also commercially available from BASF. This surfactant may comprise a hydrophobic portion such as fatty acid glycerol polyglycol esters, and a hydrophilic portion such as polyethylene glycols and glycerol ethoxylate, both of which are reported included in CREMOPHOR® RH 40.

The cosurfactant, also as noted above, may comprise a range of substances capable of performing the functions of the cosurfactant. In some presently preferred embodiments, the HLB range is about 5, and in others it is about 6. Again, the specific value may differ, depending, for example, on the specific application and system, provided the desired solubility and clarity are achieved.

A presently preferred cosurfactant comprises a propylene glycol monocaprylate, preferably comprising about 60% to about 90% monoesters. An example of such a cosurfactant that comprises about 60% monoesters would be CAPRYOL® PGMC, commercially available from Gattefosse, S. A. of Saint-Priest Cedex, France (“Gattefosse”). Another illustrative cosurfactant according to presently preferred embodiments of the invention, and one that contains about 90% monoesters, would be CAPRYOL® 90.

In presently preferred embodiments, the surfactant comprises about 30% to 70% of the combined mass of the surfactant and cosurfactant. In embodiments that are particularly preferred at present, the surfactant constitutes about 47% of the combined mass of the surfactant and cosurfactant. Similarly, in presently preferred embodiments, the cosurfactant comprises about 30% to 70% of the combined mass. In particularly preferred at present, the cosurfactant constitutes about 53% of the combined mass of the two.

In the presently preferred embodiments according to these aspects of the invention, the microemulsion composition is free, or substantially free, of a lipid phase. For example, the microemulsion composition has an HLB value of 4 or less. If there is a lipid phase, preferably it is less than 10%, by weight or volume, or the microemulsion composition.

In accordance with another aspect of the invention, a composition is provided for oral administration to a subject. This oral administration may comprise peroral administration, sublingual administration, or other oral administration forms.

The composition according to this aspect of the invention comprises a surfactant having a hydrophilic-lipophilic balance of between about 12 and 16, a cosurfactant having a hydrophilic-lipophilic balance of between about 5 and 6, and an active ingredient, wherein the composition comprises a microemulsion when added to water.

The surfactant and cosurfactant may be and preferably are as described herein for the previously described aspects of the invention and embodiments thereof.

The active ingredient may be any one or combination of a variety of substances. In view of the advantages of offered by various aspects of the invention and its preferred embodiments, however, this aspect of the invention is particularly well suited to active ingredients that otherwise do not readily lend themselves to oral administration. For example, in accordance with a preferred embodiment of this aspect of the invention, the surfactant, the cosurfactant and the active ingredient have a combined mass, e.g., which typically will be the sum of their individual masses. The active ingredient or amount of active ingredient according to this embodiment is such that it remains at least partially undissolved in an amount of vegetable oil that is equal to the combined mass. In selecting the active ingredient or the amount thereof for a particular surfactant, cosurfactant, active ingredient system, for example, one may take an amount of vegetable oil, e.g., soy bean oil, that is equal in mass to the mass of the planned system (surfactant, cosurfactant and active ingredient), and select the active ingredient or amount thereof so that it remains at least partially undissolved in that amount of oil. Systems according to this aspect of the invention may be especially well suited for cases where the amount of active ingredient that one wishes to deliver cannot simply be dissolved in vegetable oil, for example, in which case the high solubilizing ability of microemulsion formulations according to this aspect of the invention may be of especially high value. This, however, is not necessarily limiting. There could be cases, for instance, where the amount used can dissolve in a same amount of vegetable oil, but microemulsion formulations described here as being preferred can provide enhanced bioavailability.

The active ingredient according to this aspect of the invention also may be selected so that it has a low solubility in water, e.g., of no more than about 10 μg/ml.

This aspect of the invention also enables one to administer the composition, for example, in convenient capsule form, wherein the active ingredient consists of a single dosage of at least about 5 mg. According to presently preferred embodiments, the active ingredient, when administered alone, may have a bioavailability of less than about 50%, and even less than about 10%. The microemulsion of the composition according to this aspect can provide sufficiently good solubilization and release to enable adequate bioavailability at these relatively low absorption active ingredients.

Preferred compositions according to this aspect of the invention also can be used with active ingredients that have a molecular weight of about 200 to 1,000, and/or a melting point of about 60° C. to about 250° C.

Examples of presently preferred active ingredients are many, but to provide a few illustrative examples, would include androst-1-ene-3-one-17b-ethylcarbonate, androst-4-ene-3,17b-di(ethylcarbonate), forskolin 1,9-carbonate, sclareolide, androst-5-ene-7-one-3,17b-(di)ethylcarbonate), and the like.

In accordance with another aspect of the invention, a composition is provided for oral administration to a subject. The composition comprises a surfactant having a hydrophilic-lipophilic balance of between about 12 and 16, and a cosurfactant having a hydrophilic-lipophilic balance of between about 5 and 6, and an active ingredient comprising androst-1-ene-3-one-17b-ethylcarbonate, wherein the composition comprises a microemulsion when added to water.

In accordance with another aspect of the invention, a composition is provided for oral administration to a subject, wherein the composition comprises a surfactant having a hydrophilic-lipophilic balance of between about 12 and 16, and a cosurfactant having a hydrophilic-lipophilic balance of between about 5 and 6, and an active ingredient comprising androst-4-ene-3,17b-di(ethylcarbonate), wherein the composition comprises a microemulsion when added to water.

In accordance with yet another aspect of the invention, a composition is provided for oral administration to a subject, wherein the composition comprises a surfactant having a hydrophilic-lipophilic balance of between about 12 and 16, a cosurfactant having a hydrophilic-lipophilic balance of between about 5 and 6, and an active ingredient comprising androst-1-ene-3-one-17b-ethylcarbonate and androst-4-ene-3,17b-di(ethylcarbonate), wherein the composition comprises a microemulsion when added to water.

In each of the three aspects of the invention as described immediately above, the surfactant and cosurfactant may be as described herein for all of the previously described aspects of the invention and related embodiments. In presently preferred embodiments according to these three aspects of the invention, the surfactant comprises a polyoxyl castor oil, and preferably glycerol polyethylene glycol ricinoleate, for example, such as CREMOPHR® EL. Alternatively, or in addition, the surfactant may comprise glycerol polyethylene glycol oxystearate, for example, in the form of CREMOPHOR® RH 40. Alternatively or in addition, the surfactant may comprise polyethylene glycol-15-hydroxystearate, for example, such as SOLUTOL® HS 15, commercially available from BASF.

In accordance with presently preferred embodiments of these three aspects of the invention, the cosurfactant may comprise propylene glycol monocaprylate, for example, in the form of CYPRYOL® PGMC. Alternatively, or in addition, the cosurfactant may comprise CYPRYOL® 90.

The surfactant in these presently preferred embodiments constitutes about 40% of the combined mass or volume (i.e., of the surfactant plus cosurfactant) and the cosurfactant preferably constitutes about 60% of the combined mass or volume. In particular preferred embodiments at present, the surfactant constitutes about 47% of the combined mass and the cosurfactant comprises about 53% of the combined mass.

In accordance with still another aspect of the invention, a composition is provided for oral administration to a subject. The composition comprises a surfactant having a hydrophilic-lipophilic balance of between about 12 and 16, a cosurfactant having a hydrophilic-lipophilic balance of between about 5 and 6, and an active ingredient comprising a forskolin 1α,9α-carbonate, wherein the composition comprises a microemulsion when added to water.

Forskolin, sometimes referred to as forskohlin, and also known as 8,13-epoxy-1α,6β,7β,9α-tetrahydroxylabd-14-en-11-one 7β-acetate, is a diterpenoid compound having the following chemical structure: 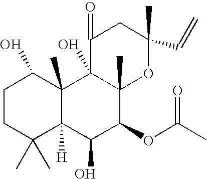
The numbering system for the carbon skeleton is well known to those in the art, and is disclosed, for example, in U.S. Pat. No. 4,088,659.

Forskolin is generally obtained by extraction from the plant known as coleus forskohlii. Traditional Ayurvedic medicine has used herbal preparations from coleus forskohlii for various therapeutic and medicinal purposes, including the treatment of heart and lung diseases, intestinal spasms, insomnia, convulsions, uterine cramps, painful urination, angina, and hypertension.

Forskolin is also reported to possess therapeutic and medicinal properties for treating many of the infirmities conventionally treated by coleus forskohlii. In addition, forskolin also reportedly has been used for other therapeutic purposes, such as ophthalmic (for lowering eye pressure and reducing the risk of glaucoma), asthma and other allergic conditions, and psoriasis, to name a few. Forskolin also has been proposed for clinical applications including weight-loss programs, hypothyroidism, malabsorption and digestive disorders, depression, prevention of cancer metastases, and immune system enhancement.

The forskolin 1α,9α-carbonate compound comprises a skeletal structure of 8,13-epoxy-1α,6β,7β,9α-tetrahydroxylabd-14-en-11-one. The forskolin 1α,9α-carbonate compound comprises a skeletal structure including carbon atoms at the 1 position, 6 position, 7 position, and 9 position, respectively. Appended to the 1-position carbon, 6-position carbon, 7-position carbon, and the 9-position carbon are a 1-position oxygen, 6-position oxygen, 7-position oxygen, and a 9-position oxygen, respectively. These oxygen atoms are also referred to herein as a 1-position hydroxy oxygen, a 6-position hydroxy oxygen, a 7-position hydroxy oxygen, and a 9-position hydroxy oxygen, respectively. The term “hydroxy oxygen” is to some extent a misnomer as used in this application, inasmuch as the forskolin 1α,9α-carbonate compound according to this invention does not necessarily include hydrogen atoms respectively appended directly to the 1-position, 6-position, 7-position, and 9-position hydroxy oxygen atoms. For example, forskolin 1α,9α-carbonate has carbonyl groups appended to the 1 and 9 position oxygen atoms.

The forskolin 1α,9α-carbonate compound according to this aspect of the invention further comprises a carbonyl group (—C(O)—) linking the 1-position hydroxy oxygen and the 9-position hydroxy oxygen to one another to form a carbonate ester ring. Preferably, the carbonate ester ring comprises, clockwise, the carbonyl carbon, the 9-position hydroxy oxygen, the 9-position carbon, a 10-position carbon, the 1-position carbon, and the 1-position hydroxy oxygen.

A hydrogen atom is appended to the 6-position hydroxy oxygen, i.e., forming a hydroxyl group. The 7-position hydroxy oxygen can be appended to two different constituents, and these constituents thus may be represented by an —R, as shown in Formula I below. 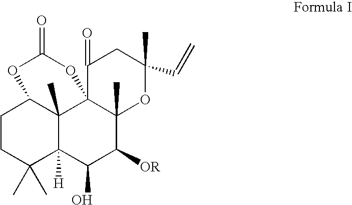
In one presently preferred embodiment, R is an acetyl group (—C(O)CH₃), as shown 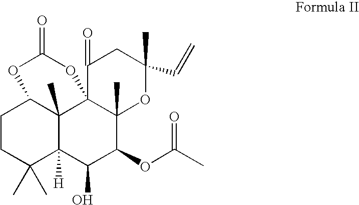
in Formula II below.

In another presently preferred embodiment, R is a hydrogen atom or proton. This compound is also referred to herein as 7-deacetyl forskolin 1α,9α-carbonate, or simply “7-DAF.”

In accordance with this aspect of the invention, the surfactant and cosurfactant may be as described herein above for the previously described aspects of the invention and related preferred embodiments.

In accordance with still another aspect of the invention, a composition is provided for oral administration to a subject. The composition according to this aspect comprises a surfactant having a hydrophilic-lipophilic balance of between about 12 and 16, a cosurfactant having a hydrophilic-lipophilic balance of between about 5 and 6, and an active ingredient comprising sclareolide, wherein the composition comprises a microemulsion when added to water.

In accordance with yet another aspect of the invention, a composition is provided for oral administration to a subject. The composition comprises a surfactant having a hydrophilic-lipophilic balance of between about 12 and 16, a cosurfactant having a hydrophilic-lipophilic balance of between about 5 and 6, and an active ingredient comprising androst-5-ene-7-one-3,17b-(di)ethylcarbonate), wherein the composition comprises a microemulsion when added to water.

In accordance with yet another aspect of the invention, a method is provided for orally administering a composition to a subject. The comprises providing the composition, wherein the composition comprises a surfactant having a hydrophilic-lipophilic balance of between about 12 and 14, a cosurfactant having a hydrophilic-lipophilic balance of between about 5 and 6, and an active ingredient, and wherein the composition comprises a microemulsion when added to water. The method further comprises administering the composition to the subject orally.

The surfactant, cosurfactant and active ingredient may be as described herein above for the previously described aspects and embodiments of the invention.

In each of the aforementioned preferred embodiments, the composition preferably is added to water, i.e., is used as a component with water, wherein the composition forms a microemulsion. The microemulsion preferably is contained in a capsule.

Additional advantages and modifications will readily occur to those skilled in the art. Therefore, the invention in its broader aspects is not limited to the specific details, representative devices and methods, and illustrative examples shown and described. Accordingly, departures may be made from such details without departing from the spirit or scope of the general inventive concept as defined by the appended claims and their equivalents.

What is claimed is:

1. A microemulsion composition comprising:
a surfactant having a hydrophilic-lipophilic balance of between about 12 and about 16; and
a cosurfactant having a hydrophilic-lipophilic balance of between about 5 and about 6.

2. A microemulsion composition as recited in claim 1, wherein the surfactant has a hydrophilic-lipophilic balance of about 12 to about 14.

3. A microemulsion composition as recited in claim 1, wherein the surfactant has a hydrophilic-lipophilic balance of about 14 to about 16.

4. A microemulsion composition as recited in claim 1, wherein the surfactant has a hydrophilic-lipophilic balance of about 14 to about 15.

5. A microemulsion composition as recited in claim 1, wherein the surfactant has a hydrophilic-lipophilic balance of about 14.

6. A microemulsion composition as recited in claim 1, wherein the surfactant has a hydrophilic-lipophilic balance of about 15.

7. A microemulsion composition as recited in claim 1, wherein the surfactant comprises a polyoxyl castor oil.

8. A microemulsion composition as recited in claim 7, wherein the surfactant comprises glycerol polyethylene glycol ricinoleate.

9. A microemulsion composition as recited in claim 7, wherein the surfactant comprises glycerol polyethylene glycol oxystearate.

10. A microemulsion composition as recited in claim 7, wherein the surfactant comprises fatty acid esters of polyethylene glycol.

11. A microemulsion composition as recited in claim 7, wherein the surfactant comprises free polyethylene glycols.

12. A microemulsion composition as recited in claim 7, wherein the surfactant comprises ethoxylated glycerol.

13. A microemulsion composition as recited in claim 1, wherein the surfactant comprises polyethylene glycol-15-hydroxystearate.

14. A microemulsion composition as recited in claim 1, wherein the cosurfactant has a hydrophilic-lipophilic balance of about 5.

15. A microemulsion composition as recited in claim 1, wherein the cosurfactant has a hydrophilic-lipophilic balance of about 6.

16. A microemulsion composition as recited in claim 1, wherein the cosurfactant comprises propylene glycol monocaprylate.

17. A microemulsion composition as recited in claim 16, wherein the propylene glycol monocaprylate comprises about 60% to about 90% monoesters.

18. A microemulsion composition as recited in claim 17, wherein the propylene glycol monocaprylate comprises about 60% monoesters.

19. A microemulsion composition as recited in claim 17, wherein the propylene glycol monocaprylate comprises about 90% monoesters.

20. A microemulsion composition as recited in claim 1, wherein:
the surfactant and the cosurfactant have a combined mass;
the surfactant comprises about 30% to 70% of the combined mass; and
the cosurfactant comprises about 30% to 70% of the combined mass.

21. A microemulsion composition as recited in claim 1, wherein:
the surfactant and the cosurfactant have a combined mass; and
the surfactant comprises about 47% of the combined mass.

22. A microemulsion composition as recited in claim 1, wherein:
the surfactant and the cosurfactant have a combined mass; and
the cosurfactant comprises about 53% of the combined mass.

23. A microemulsion composition as recited in claim 1, wherein the microemulsion composition is free of a lipid phase.

24. A microemulsion composition as recited in claim 1, wherein the microemulsion composition is substantially free of a lipid phase.

25. A composition for oral administration to a subject, the composition comprising:
a surfactant having a hydrophilic-lipophilic balance of between about 12 and 16;
a cosurfactant having a hydrophilic-lipophilic balance of between about 5 and 6; and
an active ingredient, wherein the composition comprises a microemulsion when added to water.

26. A composition for oral administration to a subject, the composition comprising:
a surfactant having a hydrophilic-lipophilic balance of between about 12 and 16;
a cosurfactant having a hydrophilic-lipophilic balance of between about 5 and 6; and
an active ingredient comprising androst-1-ene-3-one-17b-ethylcarbonate, wherein the composition comprises a microemulsion when added to water.

27. A composition for oral administration to a subject, the composition comprising:
a surfactant having a hydrophilic-lipophilic balance of between about 12 and 16;
a cosurfactant having a hydrophilic-lipophilic balance of between about 5 and 6; and
an active ingredient comprising androst-4-ene-3,17b-di(ethylcarbonate), wherein the composition comprises a microemulsion when added to water.

28. A composition for oral administration to a subject, the composition comprising:
a surfactant having a hydrophilic-lipophilic balance of between about 12 and 16;
a cosurfactant having a hydrophilic-lipophilic balance of between about 5 and 6; and
an active ingredient comprising androst-1-ene-3-one-17b-ethylcarbonate and androst-4-ene-3,17b-di(ethylcarbonate), wherein the composition comprises a microemulsion when added to water.

29. A composition as recited in claim 28, wherein the surfactant comprises a polyoxyl castor oil.

30. A composition as recited in claim 28, wherein the surfactant comprises glycerol polyethylene glycol ricinoleate.

31. A composition as recited in claim 28, wherein the surfactant comprises glycerol polyethylene glycol oxystearate.

32. A composition as recited in claim 28, wherein the surfactant comprises polyethylene glycol-15-hydroxystearate.

33. A composition as recited in claim 28, wherein the cosurfactant comprises propylene glycol monocaprylate.

34. A composition as recited in claim 28, wherein the surfactant and cosurfactant having a combined volume, the surfactant constituting about 40% of the combined volume and the cosurfactant comprising about 60% of the combined volume.

35. A composition as recited in claim 28, wherein the surfactant and cosurfactant having a combined mass, the surfactant constituting about 47% of the combined mass and the cosurfactant comprising about 53% of the combined mass.

36. A composition for oral administration to a subject, the composition comprising:
a surfactant having a hydrophilic-lipophilic balance of between about 12 and 16;
a cosurfactant having a hydrophilic-lipophilic balance of between about 5 and 6; and
an active ingredient comprising a forskolin 1α,9α-carbonate, wherein the composition comprises a microemulsion when added to water.

37. A composition for oral administration to a subject, the composition comprising:
a surfactant having a hydrophilic-lipophilic balance of between about 12 and 16;
a cosurfactant having a hydrophilic-lipophilic balance of between about 5 and 6; and
an active ingredient comprising sclareolide, wherein the composition comprises a microemulsion when added to water.

38. A composition for oral administration to a subject, the composition comprising:
a surfactant having a hydrophilic-lipophilic balance of between about 12 and 16;
a cosurfactant having a hydrophilic-lipophilic balance of between about 5 and 6; and
an active ingredient comprising androst-5-ene-7-one-3,17b-(di)ethylcarbonate), wherein the composition comprises a microemulsion when added to water.

39. A method for orally administering a composition to a subject, the method comprising:
providing the composition, wherein the composition comprises a surfactant having a hydrophilic-lipophilic balance of between about 12 and 14, a cosurfactant having a hydrophilic-lipophilic balance of between about 5 and 6, and an active ingredient, and adding water to form a microemulsion; and
administering the composition to the subject orally.
